(19) United States
(12) Patent Application Publication
(10) Pub. No.: US 2026/0265236 A1
(43) Pub. Date: Sep. 10, 2026

(54) PROCESS FOR PREPARATION OF PYROXASULFONE AND INTERMEDIATES THEREOF

(71) Applicant: UPL LIMITED, Mumbai (IN)

(72) Inventors: Prashant Vasant Kini, Mumbai (IN); Sopan Nagnath Gandhale, Mumbai (IN); Sandip Sahebrao Gulve, Mumbai (IN); Vijay Mahajan, Mumbai (IN); Jitendra Vitthal Bhosale, Mumbai (IN)

(73) Assignee: UPL LIMITED, Mumbai (IN)

(21) Appl. No.: 19/166,351

(22) PCT Filed: Mar. 19, 2024
(86) PCT No.: PCT/IB2024/052638

§ 371 Date: Sep. 17, 2025

(30) Foreign Application Priority Data
Mar. 20, 2023 (IN) 202321018910

(51) Int. Cl. C07D 413/12; C07D 231/20

(52) U.S. Cl. CPC C07D 413/12; C07D 231/20

(57) ABSTRACT

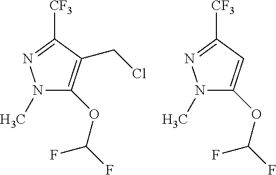 [SMILES: Cn1nc(C(F)(F)F)c(CCl)c1OC(F)F ; Cn1nc(C(F)(F)F)cc1OC(F)F]

The present invention relates to a process for preparation of 1-methyl-(3-trifluoromethyl)-1H-pyrazole compounds of formula (I) and (III), which are useful as intermediates for agrochemicals.

### FIELD OF THE INVENTION

The present invention relates to process for preparation of 1-methyl-(3-trifluoromethyl)-1H-pyrazole compounds useful as production intermediates for agrochemicals.

### BACKGROUND OF THE INVENTION

1-methyl-(3-trifluoromethyl)-1H-pyrazole compounds such as 4-(chloromethyl)-5-(difluoromethoxy)-1-methyl-(3-trifluoromethyl)-1H-pyrazole represented by compound of formula (I) and 5-(difluoromethoxy)-1-methyl-(3-trifluoromethyl)-1H-pyrazole represented by compound of formula (III),

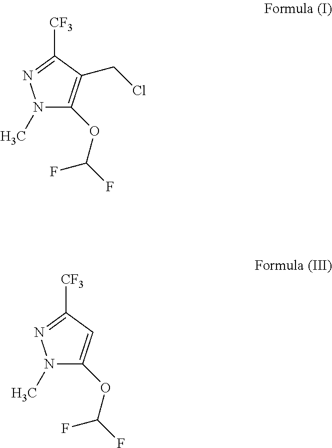 [SMILES: Cn1nc(C(F)(F)F)c(CCl)c1OC(F)F ; Cn1nc(C(F)(F)F)cc1OC(F)F]

are key intermediates useful for the preparation of pyroxasulfone, a herbicide belonging to the group pyrazolium. Pyroxasulfone is chemically known as 3-[5-(difluoromethoxy)-1-methyl-3-(trifluoromethyl) pyrazol-4-ylmethylsulfonyl]-4,5-dihydro-5,5-dimethyl-1,2-oxazole. It is a pre-emergence herbicide that inhibits the biosynthesis of very long chain fatty acids. It can be used to effectively control grass and broad leaf weeds in corn, soybean, and wheat fields. Few processes for preparation of these intermediates are known in the art. However, the processes known in prior art suffer from several drawbacks.

U.S. Pat. No. 7,256,298 discloses a process for preparation of compound of formula (I) by reacting compound of formula (III) with a chlorinating agent and paraformaldehyde in presence of an organic solvent.

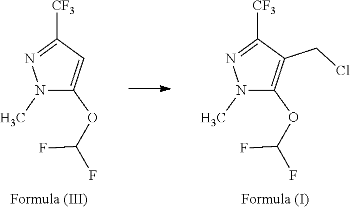 [SMILES: Cn1nc(C(F)(F)F)cc1OC(F)F ; Cn1nc(C(F)(F)F)c(CCl)c1OC(F)F]

It is well known that at industrial scale due to environmental concerns, minimal use of organic solvents is preferred. It is most desirable to avoid use of organic solvents where and when possible, to make the process environment friendly and economical. Also, the yield of compound of formula (I) obtained in said process patent is as low as 50%. Hence there remains a scope for improvement in said process wherein above discussed drawbacks are overcome.

Further, WO2022000603 discloses a process for preparation of compound of formula (I) in example 1, wherein compound of formula (I) is prepared by converting compound of formula (II) to compound of formula (IIa) by simultaneously carrying out hydroxymethylation and difluoromethylation followed by chlorination of compound of formula (IIa) to obtain compound of formula (I). Schematic representation of said reaction is as shown in scheme (I):

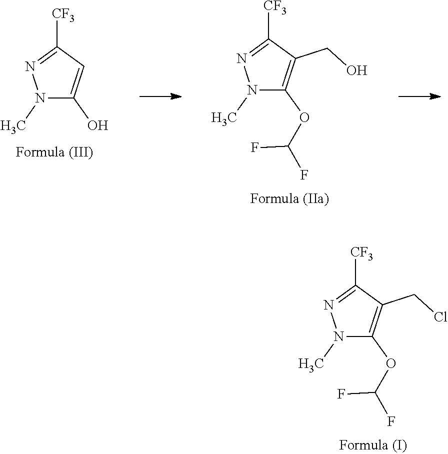 [SMILES: Cn1nc(C(F)(F)F)cc1O ; Cn1nc(C(F)(F)F)c(CO)c1OC(F)F ; Cn1nc(C(F)(F)F)c(CCl)c1OC(F)F]

The simultaneous reactions carried out in the process provided in said patent publication might lead to formation of multiple by-products. Separation of these by-products from compound of formula (I) becomes an essential step, as the purity of key intermediate have indirect impact on purity and yield of final product i.e., pyroxasulfone.

Therefore, the inventors of the present invention have skillfully designed a process to overcome all above drawbacks of processes provided in prior art and have developed a simple and environment friendly process for preparation of compound of formula (I) and compound of formula (III) with high yield.

### OBJECTIVES OF THE INVENTION

It is an objective of the present invention to provide a process for preparation of a compound of formula (III).

It is another objective of the present invention to provide an improved, simple, cost-effective and industrially viable process for preparation of compound of formula (I).

It is another objective of the present invention to provide a process for preparation of compound of formula (I) having high yield and purity.

### SUMMARY OF THE INVENTION

In an aspect of the present invention, there is provided a process for preparation of a compound of formula (III), the process comprising: reacting a compound of formula (II) with chlorodifluoromethane at a temperature less than about 30° C. to obtain a compound of formula (III).

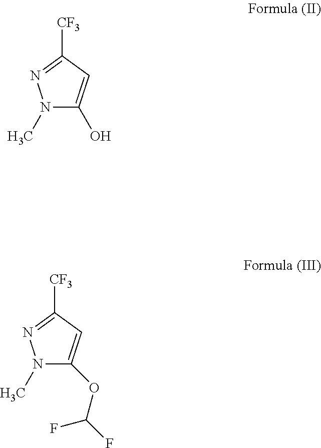

In an embodiment, there is provided a process for preparation of a compound of formula (III), the process comprising reacting a compound of formula (II) with chlorodifluoromethane in presence of a base and a solvent at a temperature less than about 30° C. to obtain a compound of formula (III).

In an aspect of the present invention, there is provided a process for preparation of a compound of formula (I), the process comprising reacting a compound of formula (III) with paraformaldehyde and a chlorinating agent in absence of an organic solvent to obtain a compound of formula (I).

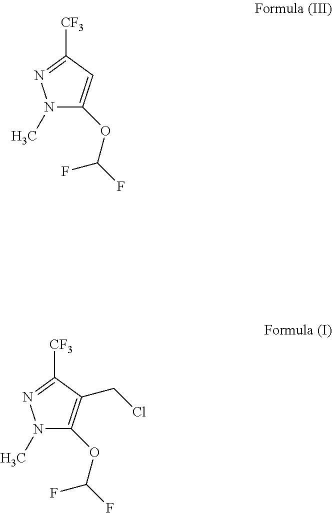 [SMILES: Cn1nc(C(F)(F)F)cc1OC(F)F ; Cn1nc(C(F)(F)F)c(CCl)c1OC(F)F]

In an aspect of the present invention, there is provided a process for preparation of compound of formula (I), the process comprising:
(a) reacting a compound of formula (II) with chlorodifluoromethane at a temperature less than about 30° C. to obtain a compound of formula (III); and

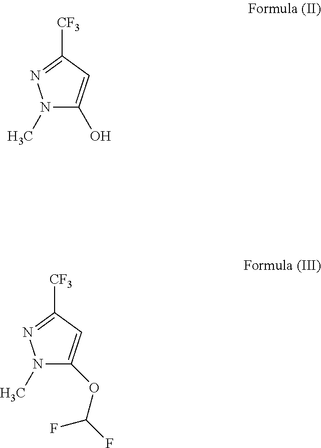

(b) reacting the compound of formula (III) with paraformaldehyde and a chlorinating agent in absence of an organic solvent to obtain a compound of formula (I).

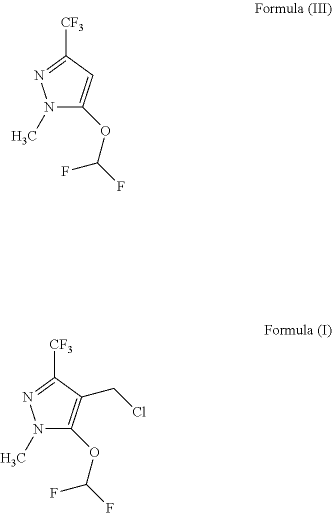 [SMILES: Cn1nc(C(F)(F)F)cc1OC(F)F ; Cn1nc(C(F)(F)F)c(CCl)c1OC(F)F]

In an embodiment, there is provided a process for preparation of pyroxasulfone of formula (VII)

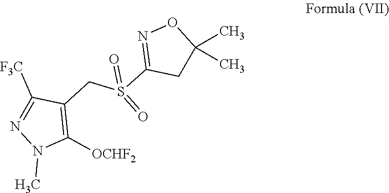 [SMILES: Cn1nc(C(F)(F)F)c(CS(=O)(=O)C2=NOC(C)(C)C2)c1OC(F)F]

using compound of formula (III) prepared according to the present invention.

In an embodiment, there is provided a process for preparation of pyroxasulfone of formula (VII)

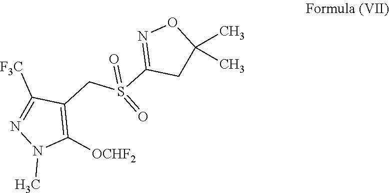 [SMILES: Cn1nc(C(F)(F)F)c(CS(=O)(=O)C2=NOC(C)(C)C2)c1OC(F)F]

using compound of formula (I) prepared according to the present invention.

### DETAILED DESCRIPTION OF THE INVENTION

Those skilled in art will be aware that invention described herein is subject to variations and modifications other than those specifically described. It is to be understood that the invention described herein includes all such variations and modifications. The invention also includes all such steps, features, compositions and methods referred to or indicated in this specification, individually or collectively, and any and all combinations of any two or more said steps or features.

For convenience, before further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances. The terms used herein are defined as follows.

The term “purity” means purity as determined by HPLC (“High Pressure Liquid Chromatography”).

As used herein, the terms “compound of formula (I)” refers to 4-(chloromethyl)-5-(difluoromethoxy)-1-methyl-(3-trifluoromethyl)-1H-pyrazole; and the terms are used interchangeably.

As used herein, the terms “compound of formula (II)’ refers to 1-methyl-3-(trifluoromethyl)-1H-pyrazol-5-ol; and the terms are used interchangeably.

As used herein, the terms “compound of formula (III)” refers to 5-(difluoromethoxy)-1-methyl-(3-trifluoromethyl)-1H-pyrazole; and the terms are used interchangeably.

The present disclosure is not to be limited in scope by the specific embodiments described herein, which are intended for the purposes of exemplification only.

In an aspect of the present invention, there is provided a process for preparation of a compound of formula (III), the process comprising: reacting a compound of formula (II) with chlorodifluoromethane at a temperature less than about 30° C. to obtain a compound of formula (III).

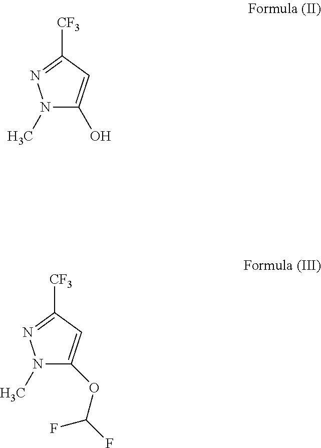

The compound of formula (II) i.e., 1-methyl-3-(trifluoromethyl)-1H-pyrazol-5-ol used as starting material is prepared according to process known in prior art.

In an embodiment, there is provided a process for preparation of a compound of formula (III), the process comprising reacting a compound of formula (II) with chlorodifluoromethane in presence of a base and a solvent at temperature less than about 30° C. to obtain a compound of formula (III).

In an embodiment, the amount of chlorodifluoromethane used is in a range from about 0.5 to about 5 moles with respect to compound of formula (II). In an embodiment, the amount of chlorodifluoromethane used is in a range from about 1 to about 3 moles with respect to compound of formula (II). In an embodiment, the amount of chlorodifluoromethane used is in a range from about 1 to about 2 moles with respect to compound of formula (II).

In an embodiment, the base used is selected from organic or inorganic base.

In an embodiment, the base used is organic base selected from, but not limited to, an organic primary, secondary or tertiary amine. In an embodiment, the organic base is selected from quaternary ammonia and amine cations including ammonium, tetramethylammonium, tetraethylammonium, methylamine, dimethylamine, trimethylamine, triethylamine, diethylamine, ethylamine, ethylenediamine, ethanolamine, diethanolamine, piperidine, piperazine, and the likes.

In an embodiment, the base used is inorganic base selected from ammonia, carbonates selected from potassium carbonate, potassium bicarbonate, sodium carbonate, sodium bicarbonate, ammonium carbonate, ammonium bicarbonate, and the likes, hydroxides selected from potassium hydroxide, sodium hydroxide, ammonium hydroxide, and the likes.

In an embodiment, the base used is sodium hydroxide. In an embodiment, the base is an aqueous solution of sodium hydroxide.

In an embodiment, the amount of base used is in a range from about 1 to about 10 moles with respect to compound of formula (II).

In an embodiment, the solvent is selected from water, nitrile solvent, C₁ to C₅ alcohols, or combination thereof. In an embodiment, the solvent is selected from water, nitrile solvent such as acetonitrile, C₁ to C₅ alcohols such as methanol, ethanol, propanol, isopropanol, and the likes.

In an embodiment, the solvent used is mixture of water and acetonitrile. In an embodiment, the solvent used is acetonitrile.

In an embodiment, the solvent used is mixture of water and isopropanol. In an embodiment, the solvent used is isopropanol.

In an embodiment, the amount of solvent used with respect to the compound of formula (II) is from about 0.5 to about 10 times.

In an embodiment, said process for preparation of compound of formula (III) is carried out at a temperature ranging from about −5° C. to about 30° C.

In an embodiment, said process for preparation of the compound of formula (III) is carried out at a temperature less than about 25° C. In an embodiment, said process for preparation of the compound of formula (III) is carried out at a temperature ranging from about −5° C. to about 25° C. In an embodiment, said process for preparation of the compound of formula (III) is carried out at a temperature ranging from about −5° C. to about 25° C. from about 10 minutes to about 5 hours.

It was observed that when said reaction was carried out at about 80° C. according to Example 1 of prior art U.S. Pat. No. 7,256,298, excess amount of chlorodifluoromethane (i.e., 5.5 to 6 moles with respect to starting material) was needed. The inventors of present invention have overcome the drawback of prior art by designing the process for preparation of said compound (III) in such a way that chlorodifluoromethane required for reaction is less than 5 moles, preferably less than 3 moles with respect to compound of formula (II).

The inventors of present invention observed that, when said reaction of compound of formula (II) with chlorodifluoromethane is carried out at temperature less than about 30° C., yield and quality of compound of formula (III) obtained was improved. Furthermore, it was also observed that moles of chlorodifluoromethane required for reaction was also reduced, which made the process economical and environment friendly.

In an embodiment, there is provided a process for preparation of compound of formula (III), the process comprising: reacting a compound of formula (II) with chlorodifluoromethane in presence of an inorganic base and acetonitrile at a temperature less than about 30° C. to obtain a compound of formula (III).

In an embodiment, the aqueous solution of inorganic base is used.

In another embodiment, the compound of formula (III) prepared according to present invention is used for preparing compound of formula (I).

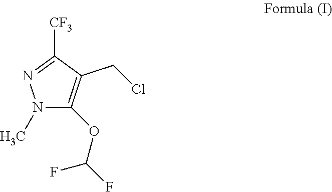 [SMILES: Cn1nc(C(F)(F)F)c(CCl)c1OC(F)F]

In yet another embodiment, the compound of formula (III) prepared according to present invention is used for preparing pyroxasulfone of formula (VII).

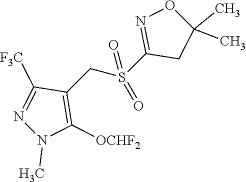 [SMILES: Cn1nc(C(F)(F)F)c(CS(=O)(=O)C2=NOC(C)(C)C2)c1OC(F)F]

In an aspect of the present invention, there is provided a process for preparation of compound of formula (I), the process comprising reacting a compound of formula (III) with paraformaldehyde and a chlorinating agent in absence of an organic solvent to obtain a compound of formula (I).

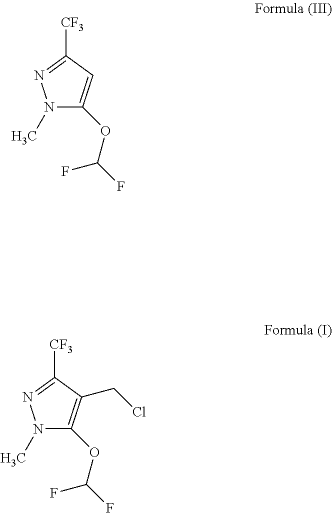 [SMILES: Cn1nc(C(F)(F)F)cc1OC(F)F ; Cn1nc(C(F)(F)F)c(CCl)c1OC(F)F]

In an embodiment, the compound of formula (III) used is prepared according to methods known in the art.

In another embodiment, the compound of formula (III) is prepared by a process comprising: reacting a compound of formula (II) with chlorodifluoromethane in presence of a base and a solvent at a temperature less than about 30° C. to obtain a compound of formula (III).

In an embodiment, the amount of paraformaldehyde used is in a range from about 1 to about 5 moles with respect to compound of formula (III).

In an embodiment, the chlorinating agent used is selected from thionyl chloride, phosphorus trichloride, sulfuryl chloride, sulfonyl chloride, chlorine, hydrochloric acid, and the like.

In an embodiment, the chlorinating agent used is hydrochloric acid. Preferably, concentrated hydrochloric acid is used.

In an embodiment, the amount of chlorinating agent used is in the range of about 1 to 15 moles with respect to compound of formula (III). In an embodiment, the amount of chlorinating agent used is in the range of about 7 to 12 moles with respect to compound of formula (III).

In an embodiment, the process for preparation of the compound of formula (I), is carried out in presence of a Lewis acid. In another embodiment, said chlorinating agent is used along with a Lewis acid. The Lewis acid used is selected from zinc chloride, titanium tetrachloride, aluminium chloride, zinc bromide, or combination thereof.

In an embodiment, the amount of Lewis acid used is in a range from about 0.5 to about 3 moles with respect to the compound of formula (III).

In an embodiment, the chlorinating agent used is hydrochloric acid, preferably concentrated hydrochloric acid; and the Lewis acid used is zinc chloride.

In an embodiment, reaction of the compound of formula (III) with paraformaldehyde and a chlorinating agent to obtain the compound of formula (I) is carried out at a temperature ranging from about 40° C. to about 100° C. Preferably, said reaction is carried out at a temperature ranging from about 50° C. to about 80° C.

In an embodiment, reaction of the compound of formula (III) with paraformaldehyde, concentrated hydrochloric acid and zinc chloride in absence of an organic solvent is carried out at a temperature ranging from about 50° C. to about 80° C.

The prior art processes provide similar reaction for preparation of the compound of formula (I) however, these processes involve use of organic solvents such as carboxylic acids or carbon disulfide, etc. Use of carboxylic acids like acetic acid as solvent may lead to formation of undesired impurity thereby negatively impacting the yield and purity of desired compound. Inventors of the present invention have developed a process for preparation of the compound of formula (I), without using any organic solvent.

In an aspect of the present invention, there is provided a process for preparation of compound of formula (I), the process comprising:
(a) reacting a compound of formula (II) with chlorodifluoromethane at a temperature less than about 30° C. to obtain a compound of formula (III); and

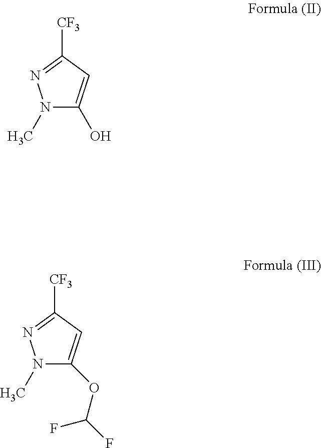

(b) reacting the compound of formula (III) with paraformaldehyde and a chlorinating agent, in absence of an organic solvent to obtain the compound of formula (I).

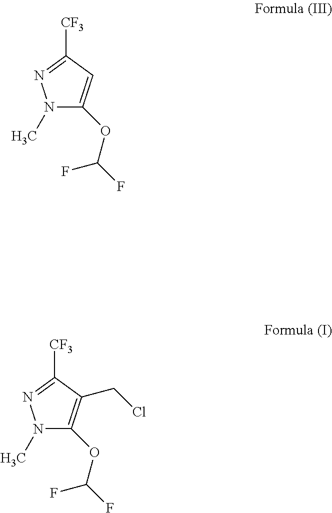 [SMILES: Cn1nc(C(F)(F)F)cc1OC(F)F ; Cn1nc(C(F)(F)F)c(CCl)c1OC(F)F]

In an embodiment, the step (a) is carried out in presence of a base and a solvent. The base use is selected from organic or inorganic base. The solvent used in step (a) is selected from nitrile solvent such as acetonitrile, alcohols such as methanol, ethanol, propanol, isopropanol, and the likes.

In an embodiment, the chlorinating agent used in step (b) is selected from thionyl chloride, phosphorus trichloride, sulfuryl chloride, sulfonyl chloride, chlorine, hydrochloric acid, and the like.

In an embodiment, the step (b) is carried out in presence of a Lewis acid. The Lewis acid used is selected from titanium tetrachloride, zinc chloride, aluminium chloride, zinc bromide, and the likes.

In an embodiment, there is provided a process for preparation of a compound of formula (I), the process comprising:
(a) reacting a compound of formula (II) with chlorodifluoromethane in presence of an inorganic base and acetonitrile at a temperature less than about 30° C. to obtain a compound of formula (III); and
(b) reacting the compound of formula (III) with paraformaldehyde and concentrated hydrochloric acid in presence of zinc chloride, and in absence of an organic solvent to obtain the compound of formula (I).

In an embodiment, the amount of chlorodifluoromethane used in step (a) is in a range from about 0.5 to about 5 moles with respect to the compound of formula (II).

In an embodiment, the inorganic base used in step (a) is selected from carbonates selected from potassium carbonate, potassium bicarbonate, sodium carbonate, sodium bicarbonate and the likes, hydroxides selected from potassium hydroxide, sodium hydroxide, ammonium hydroxide, and the likes.

In an embodiment, the step (a) is carried out at a temperature ranging from about-5° C. to about 25° C.

In an embodiment, the step (b) is carried out in aqueous medium. In an embodiment, the step (b) is carried out at a temperature ranging from about 40° C. to about 100° C. Preferably, said reaction is carried out at a temperature ranging from about 50° C. to about 80° C.

In an embodiment, the compound of formula (I) prepared according to the present invention is employed as a key intermediate in preparation of pyroxasulfone by methods known in prior art. In an embodiment, the compound of formula (III) prepared according to the present invention is employed as key intermediate for the preparation of pyroxasulfone by methods know in prior art.

In an embodiment, the compound of formula (III) prepared according to the present invention is further employed for the preparation of pyroxasulfone of formula (VII).

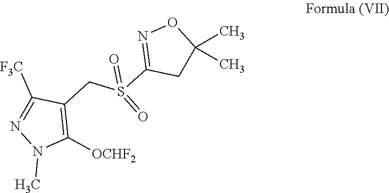 [SMILES: Cn1nc(C(F)(F)F)c(CS(=O)(=O)C2=NOC(C)(C)C2)c1OC(F)F]

In an embodiment, there is provided a process for preparation of pyroxasulfone of formula (VII) using compound of formula (III) prepared according to the present invention.

In an embodiment, there is provided a process for preparation of pyroxasulfone of formula (VII):

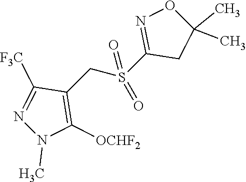 [SMILES: Cn1nc(C(F)(F)F)c(CS(=O)(=O)C2=NOC(C)(C)C2)c1OC(F)F]

using the compound of formula (I) prepared according to the present invention.

In an embodiment, there is provided a process for preparation of pyroxasulfone of formula (VII) using a compound of formula (I) prepared according to the present invention, wherein the process comprises:
(a) condensing the compound of formula (I) or a derivative thereof with a suitable isoxazoline compound to obtain a compound of formula (VI); and
(b) oxidising the compound of formula (VI) to obtain pyroxasulfone of formula (VII).

In an embodiment, the compound of formula (I) derivative includes compound of formula (IV). In an embodiment, suitable isoxazoline compound includes compound of formula (V) or compound of formula (VIII).

In an embodiment, there is provided a process for preparation of pyroxasulfone of formula (VII) using a compound of formula (I) prepared according to the present invention, wherein the process comprises:
(a) condensing the compound of formula (I) with thiourea to obtain a compound of formula (IV) or its salt:

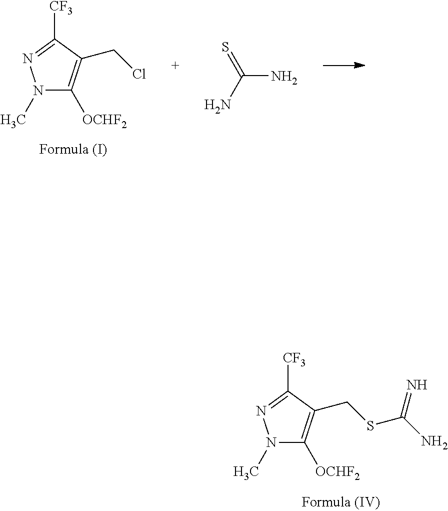

(b) condensing the compound of formula (IV) or its salt with isoxazoline compound of formula (V) to obtain the compound of formula (VI);

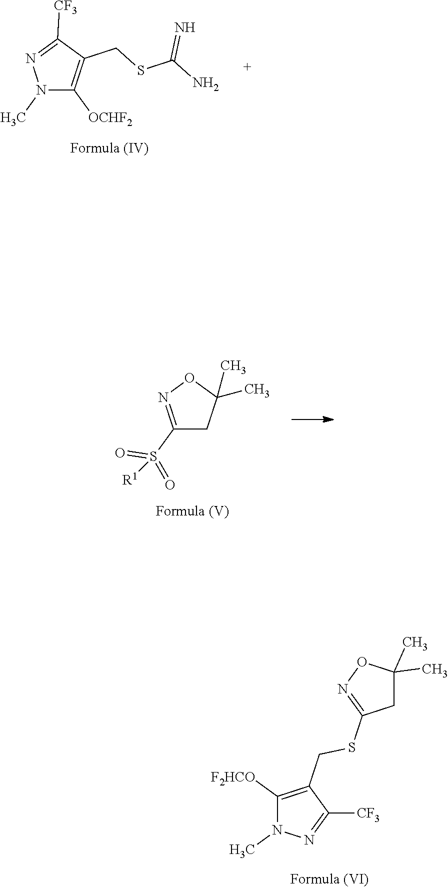

wherein R¹ is C₁ to C₅ alkyl group; and
(c) oxidising the compound of formula (VI) to obtain pyroxasulfone of formula (VII).

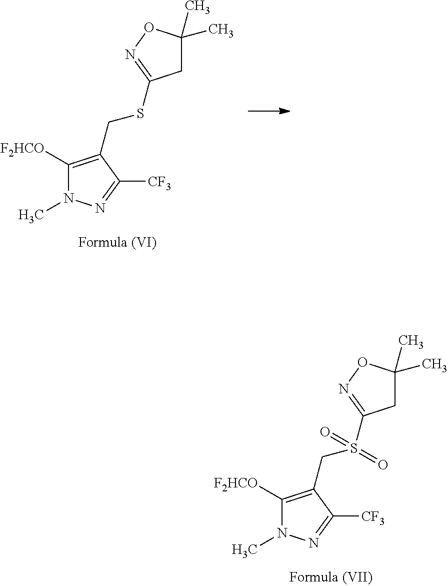 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

According to an embodiment, the compound of formula (I) is condensed with thiourea in presence of an organic solvent at a temperature ranging from about 0° C. to about 150° C. In an embodiment, the compound of formula (I) is condensed with thiourea at a temperature ranging from about 20° C. to about 100° C.

In an embodiment, the organic solvent used is selected from lower alcohol such as methanol, ethanol, isopropanol, n-propanol, butanol, tert-butanol and the like, hydrocarbons such as toluene, xylene, benzene and like, halogenated hydrocarbons such as dichloromethane, dichloroethane, chloroform and the like, ethers such as methyl tert-butyl ether, tetrahydrofuran, dioxane and the like. In an embodiment, the organic solvent used is ethanol.

According to another embodiment, the isoxazoline compound of formula (V) wherein R¹ is methyl group is used in step (b). In an embodiment, the isoxazoline compound of formula (V) is 4,5-dihydro-5,5-dimethyl-3-(methylsulfonyl) isoxazole.

According to another embodiment, the step (b) of the process comprises condensing compound of formula (IV) or its salt with isoxazoline compound of formula (V) and said step is carried out in presence of a base comprising carbonates selected from potassium carbonate, potassium bicarbonate, sodium carbonate, sodium bicarbonate and the like, hydroxides selected from potassium hydroxide, sodium hydroxide, ammonium hydroxide and the like, or alkoxides like sodium alkoxide or potassium alkoxide. In an embodiment, the base used is potassium carbonate.

According to an embodiment, the step (b) is carried out in presence of a polar solvent such as alcohols like methanol, ethanol, isopropanol, n-propanol, tert-butanol and the like, ethers such as tetrahydrofuran, 1,6-dioxane and the like, water, dimethylformamide, or combination thereof. According to an embodiment, the polar solvent comprises alcohol, water, or combination thereof.

In an embodiment, the step (b) is carried out at a temperature ranging from about 0° C. to about 150° C. In an embodiment, the step (b) is carried out at a temperature ranging from about 0° C. to about 60° C.

According to another embodiment, the step (c) of the process comprises oxidising compound of formula (VI) to obtain pyroxasulfone of formula (VII), wherein oxidation is carried out in presence of an oxidizing agent. The oxidizing agent used may be selected from organic peroxides such as m-chloroperbenzoic acid, performic acid, peracetic acid and the like; inorganic peroxides such as hydrogen peroxide, potassium permanganate, sodium periodate or potassium peroxymonosulfate and the like. In an embodiment, the oxidising agent used is hydrogen peroxide. In an embodiment, the oxidising agent used is potassium peroxymonosulfate.

According to another embodiment, the step (c) oxidation is carried out in presence of an oxidizing agent in an organic solvent to obtain the pyroxasulfone of formula (VII).

According to an embodiment, the step (c) oxidation is carried out in presence of a catalyst, preferably a metal catalyst. The catalyst used can be a metal catalyst such as tungsten catalyst, molybdenum catalyst, titanium catalyst, zirconium catalyst, or combination thereof. Preferably, the tungsten catalyst used is sodium tungstate, more preferably sodium tungstate dihydrate is used.

According to an embodiment, the oxidation step is carried out in presence of oxidising agent and a metal catalyst. According to an embodiment, the oxidation step is carried out in presence of hydrogen peroxide as an oxidising agent and sodium tungstate dihydrate as a metal catalyst.

According to an embodiment, the step (c) oxidation is carried out in presence of a metal catalyst and an acid. The acid used is an inorganic acid or organic acid. The inorganic acid like sulfuric acid, hydrochloric acid, or an organic acid such as acetic acid and formic acid.

According to an embodiment, the step (c) oxidation is carried out in presence of a suitable solvent selected from the group comprising halogenated hydrocarbon; ethers; amides; alcohols; ketones; nitriles; carboxylic acids; water or combination thereof. The organic solvent used may be selected from halogenated hydrocarbon such as dichloromethane, chloroform, dichloroethane, carbon tetrachloride, chlorobenzene, dichlorobenzene and the like; ethers such as dioxane, tetrahydrofuran (THF), dimethoxyethane, diethyl ether and the like; amides such as N,N-dimethylacetamide, N,N-dimethylformamide, N-methyl-2-pyrrolidinone and the like; alcohols such as methanol, ethanol, propanol, isopropanol, butanol, tert-butanol and the like; ketones such as acetone,2-butanone and the like: nitriles such as acetonitrile and the like; carboxylic acids like acetic acid; water, and combination thereof. In an embodiment, organic solvent used is acetic acid.

In another embodiment, there is provided a process for preparation of pyroxasulfone of formula (VII) using the compound of formula (I) prepared according to the present invention, wherein the process comprises:
(a) condensing a compound of formula (I) with an isoxazoline compound of formula (VIII) or its salt to obtain a compound of formula (VI); and

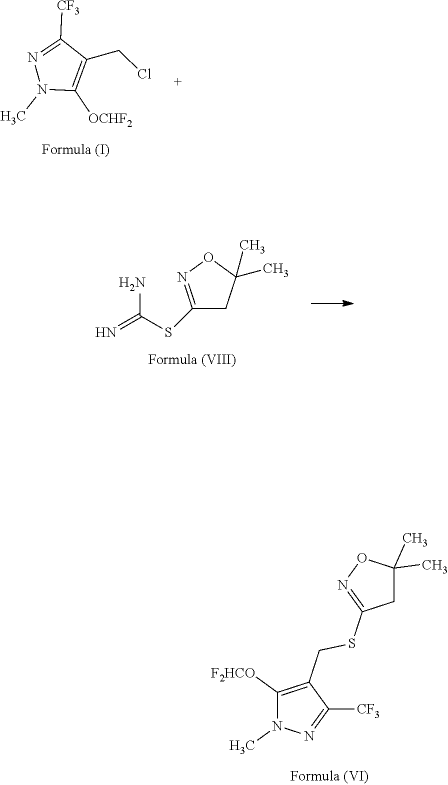 [SMILES: Cn1nc(C(F)(F)F)c(CCl)c1OC(F)F ; CC1(C)CC(SC(=N)N)=NO1 ; Cn1nc(C(F)(F)F)c(CSC2=NOC(C)(C)C2)c1OC(F)F]

(b) oxidising the compound of formula (VI) to obtain pyroxasulfone of formula (VII).

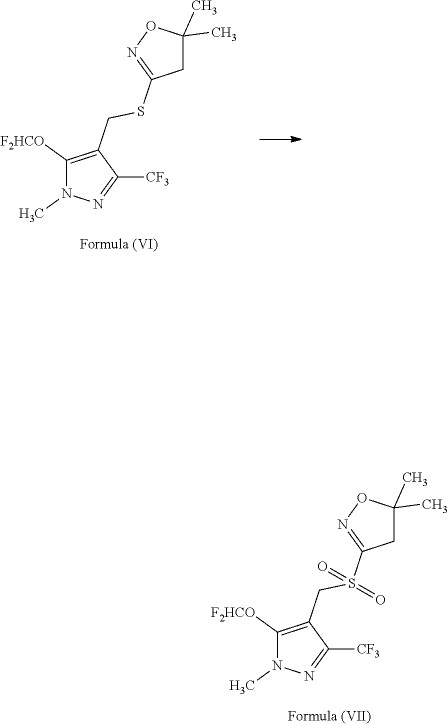 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

According to another embodiment, the step (a) of the process comprises condensing the compound of formula (I) with an isoxazoline compound of formula (VIII) or its salt carried out in presence of a base comprising carbonate selected from, but not limited to, potassium carbonate, potassium bicarbonate, sodium carbonate, sodium bicarbonate and the like, hydroxides selected from potassium hydroxide, sodium hydroxide, ammonium hydroxide and the like, or alkoxides like sodium alkoxide or potassium alkoxide.

According to another embodiment, the step (a) of the process is performed in the presence of an organic solvent, water, or combination thereof. The organic solvent in the reaction in the step (a) is selected from aromatic hydrocarbon derivatives, halogenated aliphatic hydrocarbons, alcohols, nitriles, carboxylic acid esters, ethers, ketones, amides, ureas, sulfoxides and sulfones.

According to another embodiment, the step (b) of the process comprises oxidising compound of formula (VI) to obtain pyroxasulfone of formula (VII) wherein oxidation is carried out in presence of an oxidizing agent. The oxidizing agent used may be selected from organic peroxides such as m-chloroperbenzoic acid, performic acid, peracetic acid and the like; inorganic peroxides such as hydrogen peroxide, potassium permanganate, sodium periodate or potassium peroxymonosulfate and the like.

According to another embodiment, the step (b) oxidation is carried out in presence of an oxidizing agent in an organic solvent to obtain pyroxasulfone of formula (VII).

According to an embodiment, the step (b) oxidation is carried out in presence of a catalyst, preferably a metal catalyst. The catalyst used can be a metal catalyst such as tungsten catalyst, molybdenum catalyst, titanium catalyst, zirconium catalyst or combination thereof. Preferably, the tungsten catalyst used is sodium tungstate, more preferably sodium tungstate dihydrate is used.

According to an embodiment, the oxidation step is carried out in presence of an oxidising agent and a metal catalyst. According to an embodiment, the oxidation step is carried out in presence of hydrogen peroxide as an oxidising agent and sodium tungstate dihydrate as a metal catalyst.

According to an embodiment, the step (b) oxidation is carried out in presence of a metal catalyst and an acid. The acid used is an inorganic acid or organic acid. The inorganic acid comprises sulfuric acid, hydrochloric acid, and the organic acid comprises acetic acid and formic acid.

According to an embodiment, the step (b) oxidation is carried out in presence of a suitable solvent selected from group comprising halogenated hydrocarbon; ethers; amides; alcohols; ketones; nitriles; carboxylic acids; water or combination thereof. The organic solvent used may be selected from halogenated hydrocarbon such as dichloromethane, chloroform, dichloroethane, carbon tetrachloride, chlorobenzene, dichlorobenzene and the like; ethers such as dioxane, tetrahydrofuran (THF), dimethoxyethane, diethyl ether and the like; amides such as N,N-dimethylacetamide, N,N-dimethylformamide, N-methyl-2-pyrrolidinone and the like; alcohols such as methanol, ethanol, propanol, isopropanol, butanol, tert-butanol and the like; ketones such as acetone,2-butanone and the like: nitriles such as acetonitrile and the like acetic acid; water, and combination thereof. According to an embodiment, the oxidation is carried out in presence of potassium peroxymonosulfate as an oxidising agent.

### Advantages of the Present Invention

1. The present invention provides a simple, cost-effective and industrially viable process for preparation of the compound of formula (I) having high yield and purity.
2. The present process for preparation of the compound of formula (I) is carried out in absence of an organic solvent, thereby reducing economic and environmental burden.
3. The preset process for preparation of the compound of formula (III) requires lesser amount of chlorodifluoromethane, thereby making the process eco-friendly and cost effective.

### EXAMPLES

The following examples are presented to provide what is believed to be the most useful and readily understood description of procedures and conceptual aspects of this invention. The examples provided below are merely illustrative of the invention and are not intended to limit the same to disclosed embodiments.

### Analytical Method Details:

Samples were analysed on high performance liquid chromatography (HPLC) with UV detector using Zorbax SB C-8 (250 mm×4.6 mm, 5μ).

Mobile Phase-0.1% OPA:Acetonitrile (90:10)

### Example 1: Preparation of 5-(difluoromethoxy)-1-methyl-(3-trifluoromethyl)-1H-pyrazole i.e., Compound of Formula (III)

To a solution of 200 g 1-methyl-3-(trifluoromethyl)-1H-pyrazol-5-ol i.e., the compound of formula (II) in 1017 ml acetonitrile; was added 264 ml 48% caustic solution and 134.7 g difluorochloromethane gas at about 15° C. to 20° C. and the reaction was maintained till completion. After completion of reaction, 200 ml water was added under stirring to the reaction mixture and the layers were separated. The organic layer separated was washed with 20% brine and then the solvent in organic layer was distilled out to obtain 260 g 5-(difluoromethoxy)-1-methyl-(3-trifluoromethyl)-1H-pyrazole. (Purity: 92.55% w/w)

### Example 2: Preparation of 5-(difluoromethoxy)-1-methyl-(3-trifluoromethyl)-1H-pyrazole i.e., the Compound of Formula (III)

To a solution of 100 g 1-methyl-3-(trifluoromethyl)-1H-pyrazol-5-ol i.e., the compound of formula (II) in 508 ml isopropanol; was added 132 ml 48% caustic solution and 104.1 g difluorochloromethane gas at about 10° C. to 20° C. and the reaction was maintained for 1 hour. After completion of reaction, 100 ml water was added under stirring to the reaction mixture and the layers were separated. The organic layer separated was washed with 20% brine and then the solvent in organic layer was distilled out to obtain 122.76 g 5-(difluoromethoxy)-1-methyl-(3-trifluoromethyl)-1H-pyrazole. (Purity: 95.38% w/w)

### Example 3: Preparation of 4-(chloromethyl)-5-(difluoromethoxy)-1-methyl-(3-trifluoromethyl)-1H-pyrazole i.e., Compound of Formula (I)

To a mixture of 225 g 5-(difluoromethoxy)-1-methyl-(3-trifluoromethyl)-1H-pyrazole i.e., the compound of formula (III), 86.72 g paraformaldehyde, 196.7 g zinc chloride was added with 724 ml concentrated hydrochloric acid at room temperature. The mixture was then heated to about 60° C. to 65° C. for about 5 to 7 hours. After completion of the reaction, 423 ml dichloromethane was added to the reaction mixture and layers were separated. The solvent in organic layer separated was distilled out to obtain 253 g yellow to brown liquid of 4-(chloromethyl)-5-(difluoromethoxy)-1-methyl-(3-trifluoromethyl)-1H-pyrazole i.e., the compound of formula (I). (Purity 97.50% w/w)

### Example 4: Preparation of [5-(Difluoromethoxy)-1-methyl-3-(trifluoromethyl)-1H-pyrazol-4-yl]methyl carbamimidothioate i.e., the Compound of Formula (IV)

The mixture of 88 ml ethanol, 95.6 g 4-(chloromethyl)-5-(difluoromethoxy)-1-methyl-(3-trifluoromethyl)-1H-pyrazole i.e., the compound of formula (I) and 30.26 g thiourea was heated to about 60° C. to 65° C. for about 2 to 3 hours to obtain a reaction mass comprising [5-(Difluoromethoxy)-1-methyl-3-(trifluoromethyl)-1H-pyrazol-4-yl]methyl carbamimidothioate i.e., the compound of formula (IV).

### Example 5: Preparation of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole i.e., the Compound of Formula (VI)

To the reaction mass comprising [5-(Difluoromethoxy)-1-methyl-3-(trifluoromethyl)-1H-pyrazol-4-yl]methyl carbamimidothioate i.e., compound of formula (IV) obtained in Example 4, was added 60.93 g 4,5-Dihydro-5,5-dimethyl-3-(methylsulfonyl) isoxazole i.e., the compound of formula (V) wherein R¹ is methyl group; to this mixture 347.3 ml water and 308 ml ethanol was added. To the mixture obtained was then added 121.23 g potassium carbonate and the mixture was then stirred at about 25° C. to 35° C. for about 8 to 9 hours. After completion of reaction, solvents were distilled out to obtain concentrated mass. To the concentrated mass obtained was added 78.95 ml water and the mixture was then extracted with methylene dichloride. The organic layer was separated, washed with 5% sulfuric acid solution and brine solution, and then the solvent was distilled out to obtain reaction mass comprising 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole i.e., the compound of formula (VI).

### Example 6: Preparation of Pyroxasulfone

To the reaction mass comprising 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole i.e., the compound of formula (VI) obtained in Example 5, was added 858 ml acetic acid, 4.637 g sodium tungstate dihydrate and 67 ml 50% hydrogen peroxide. Then the temperature was increased to about 50° C. to 55° C. and maintained for another 7 hours. The reaction was monitored by HPLC. After completion of the reaction, the mixture was cooled to about 25° C. to 30° C. and diluted with 246.3 ml water. The product was filtered out and washed with water and petroleum ether and dried to yield 109.9 g pyroxasulfone.

1. A process for preparation of a compound of formula (III), the process comprising:
reacting a compound of formula (II) with chlorodifluoromethane at a temperature less than about 30° C. to obtain the compound of formula (III).

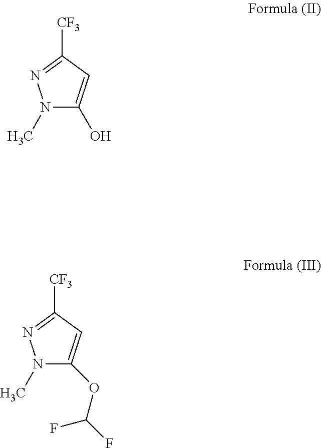

2. The process as claimed in claim 1, wherein amount of chlorodifluoromethane used is in a range from about 1 to about 3 moles with respect to the compound of formula (II).

3. The process as claimed in claim 1, wherein said reaction is carried out in presence of a base and a solvent.

4. The process as claimed in claim 3, wherein the solvent is selected from water, nitrile solvent, C1 to C5 alcohols, or combination thereof.

5. The process as claimed in claim 1, wherein said reaction is carried out at a temperature ranging from about −5° C. to about 30° C.

6. The process as claimed in claim 1, wherein the compound of formula (III) is used for preparing a compound of formula (I).

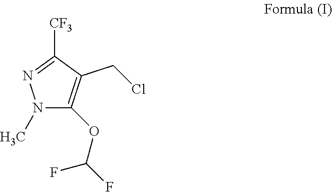 [SMILES: Cn1nc(C(F)(F)F)c(CCl)c1OC(F)F]

7. The process as claimed in claim 1, wherein the compound of formula (III) is further used for preparing pyroxasulfone of formula (VII).

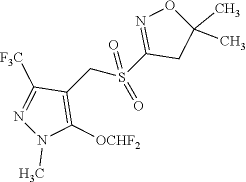 [SMILES: Cn1nc(C(F)(F)F)c(CS(=O)(=O)C2=NOC(C)(C)C2)c1OC(F)F]

8. A process for preparation of a compound of formula (I), the process comprising:
reacting a compound of formula (III) with paraformaldehyde and a chlorinating agent in absence of an organic solvent to obtain the compound of formula (I).

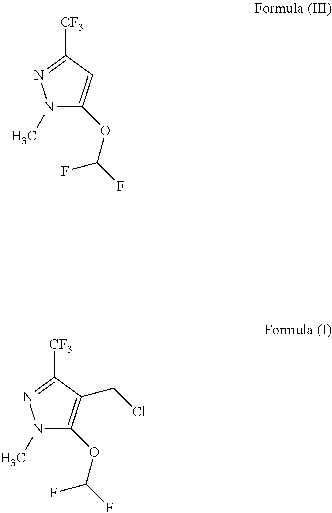 [SMILES: Cn1nc(C(F)(F)F)cc1OC(F)F ; Cn1nc(C(F)(F)F)c(CCl)c1OC(F)F]

9. The process as claimed in claim 8, wherein the chlorinating agent is selected from thionyl chloride, phosphorus trichloride, sulfuryl chloride, sulfonyl chloride, chlorine, or hydrochloric acid.

10. The process as claimed in claim 8, wherein said reaction is carried out in presence of a Lewis acid.

11. The process as claimed in claim 10, wherein the Lewis acid is selected from zinc chloride, titanium tetrachloride, aluminium chloride, or zinc bromide.

12. The process as claimed in claim 8, wherein said reaction is carried out at a temperature ranging from about 40° C. to about 100° C.

13. A process for preparation of a compound of formula (I), the process comprising:
(a) reacting a compound of formula (II) with chlorodifluoromethane at a temperature less than about 30° C. to obtain a compound of formula (III); and 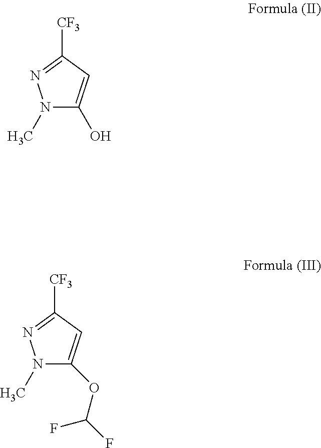
(b) reacting the compound of formula (III) with paraformaldehyde and a chlorinating agent to obtain the compound of formula (I).

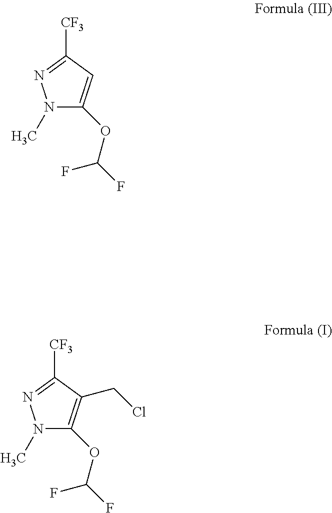 [SMILES: Cn1nc(C(F)(F)F)cc1OC(F)F ; Cn1nc(C(F)(F)F)c(CCl)c1OC(F)F]

14. The process as claimed in claim 13, wherein the process further comprises:
(a) condensing the compound of formula (I) or a derivative thereof with a suitable isoxazoline compound to obtain a compound of formula (VI); and
(b) oxidising the compound of formula (VI) to obtain pyroxasulfone of formula (VII).
